(19) United States
(12) Patent Application Publication
(10) Pub. No.: US 2026/0159792 A1
(43) Pub. Date: Jun. 11, 2026

(54) DETECTION DEVICE

(71) Applicant: Japan Display Inc., Tokyo (JP)

(72) Inventor: Tomoya TEZEN, Tokyo (JP)

(21) Appl. No.: 19/408,025

(22) Filed: Dec. 3, 2025

(30) Foreign Application Priority Data
Dec. 5, 2024 (JP) 2024-212518

(51) Int. Cl. C12M 1/34; G01N 21/25; G01N 21/27

(52) U.S. Cl. CPC C12M 1/3446; G01N 21/255; G01N 21/27; G01N 2201/067

(57) ABSTRACT

According to an aspect, a detection device includes: a light source configured to emit light; a planar optical sensor on which a plurality of optical sensors configured to detect the light from the light source are two-dimensionally arranged; an object placement member on which an object to be detected is placed between the light source and the planar optical sensor; a light controller that is provided between the object placement member and the light source and is configured to diffuse the light from the light source; and an optical member that is provided between the object placement member and the planar optical sensor and configured to limit light reaching the optical sensors.

### CROSS-REFERENCE TO RELATED APPLICATION

This application claims the benefit of priority from Japanese Patent Application No. 2024-212518 filed on Dec. 5, 2024, the entire contents of which are incorporated herein by reference.

### BACKGROUND

### 1. Technical Field

What is disclosed herein relates to a detection device.

### 2. Description of the Related Art

Devices are known that acquire an image by imaging a Petri dish in which a culture medium (e.g., agar) for culturing cultivation targets such as bacteria is formed, and detect colonies of the cultivation targets formed on the culture medium from the image (for example, Japanese Patent Application Laid-open Publication No. 2012-080802).

When using a light source and an optical sensor arranged so as to face each other with the Petri dish interposed therebetween to image the Petri dish, an uneven light distribution from the light source may affect an output of the optical sensor, disabling good detection of the colonies.

For the foregoing reasons, there is a need for a detection device capable of better detection of colonies.

### SUMMARY

According to an aspect, a detection device includes: a light source configured to emit light; a planar optical sensor on which a plurality of optical sensors configured to detect the light from the light source are two-dimensionally arranged; an object placement member on which an object to be detected is placed between the light source and the planar optical sensor; a light controller that is provided between the object placement member and the light source and is configured to diffuse the light from the light source; and an optical member that is provided between the object placement member and the planar optical sensor and configured to limit light reaching the optical sensors.

### BRIEF DESCRIPTION OF THE DRAWINGS

FIG. 1 is a diagram illustrating a main configuration of a detection device;

FIG. 2 is a diagram illustrating a configuration example of a detection area and a wiring area;

FIG. 3 is a circuit diagram illustrating a circuit configuration of an optical sensor;

FIG. 4 is a schematic diagram illustrating a configuration example of a light source;

FIG. 5 is a schematic diagram schematically illustrating a configuration example of a detection system;

FIG. 6 is a schematic diagram illustrating a relation between one detection device and an external configuration;

FIG. 7 is a schematic diagram illustrating an example of main components of the detection device and also illustrating structures of components including an object to be detected that is placed on the detection device;

FIG. 8 is a schematic plan view illustrating a case where the object to be detected, placed on a light-transmitting member, is viewed from a planar optical sensor side;

FIG. 9 illustrates explanatory views regarding effects of light diffusion by a diffusion plate;

FIG. 10 is a flowchart of processing related to operations of the detection device;

FIG. 11 is a flowchart of an initial operation;

FIG. 12 is a flowchart of a periodic operation;

FIG. 13 is a schematic diagram illustrating another example of the main components of the detection device and also illustrating the structures of the components including the object to be detected placed on the detection device;

FIG. 14 is a schematic sectional view of a dimming panel;

FIG. 15 illustrates additional explanatory views regarding the effects of the light diffusion by the dimming panel;

FIG. 16 is a flowchart of processing related to operations of a detection device according to a modification of the embodiment; and

FIG. 17 is a flowchart of processing of a post-notification operation.

### DETAILED DESCRIPTION

The following describes an embodiment of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that illustrated in a drawing that has already been discussed is denoted by the same reference numeral through the description and the drawings, and detailed description thereof may not be repeated where appropriate.

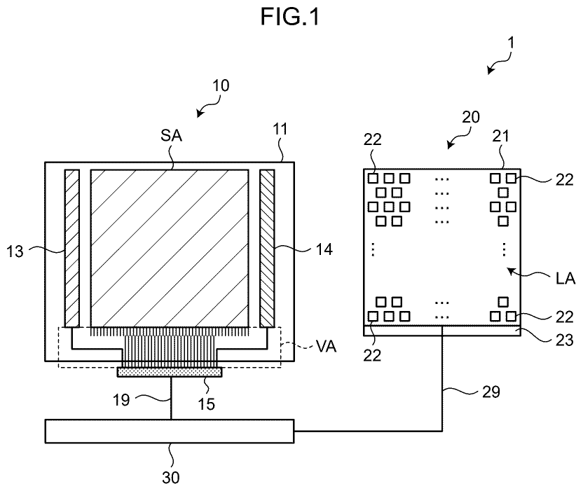

FIG. 1 is a diagram illustrating a main configuration of a detection device 1. The detection device 1 includes a planar optical sensor 10, a light source panel 20, and a control circuit 30. The planar optical sensor 10 and the light source panel 20 of the detection device 1 are coupled to the control circuit 30.

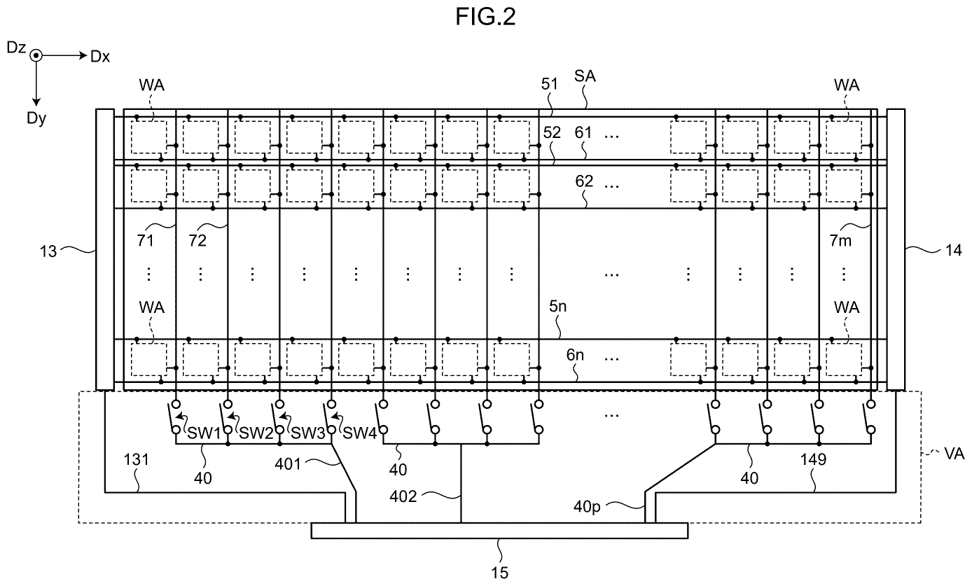

The planar optical sensor 10 is provided with a detection area SA (refer to FIG. 2) on a substrate 11. A reset circuit 13, a scan circuit 14, and a wiring area VA are provided on the substrate 11. Components on the detection area SA, the reset circuit 13, and the scan circuit 14 are coupled to a detection circuit 15 via the wiring area VA.

The light source panel 20 has a light-emitting area LA that emits light to the detection area SA. The light source panel 20 is provided with a light source 22 on a substrate 21. The light source 22 emits light. Specifically, the light source 22 includes a light-emitting elements such as a light-emitting diode (LED), and is provided in the light-emitting area LA. In the example illustrated in FIG. 1, a plurality of the light sources 22 are arranged in a staggered manner on the substrate 21, but the arrangement of the light sources 22 is not limited to this arrangement. The light sources 22 may be arranged, for example, in a matrix having a row-column configuration.

The light source panel 20 is provided with a light source drive circuit 23. Under the control of the control circuit 30, the light source drive circuit 23 controls turning on and off of each of the light sources 22 and the luminance thereof when being turned on. The light sources 22 may be provided so as to be individually controllable in light emission, or may be provided so as to emit light all together.

The control circuit 30 performs various processes related to operations of the detection device 1. Specifically, the control circuit 30 is a circuit, such as a field-programmable gate array (FPGA) that can implement a plurality of functions. The control circuit 30 may have other configurations, such as an application-specific integrated circuit (ASIC). The control circuit 30 is coupled to the light source drive circuit 23 via wiring 29 and performs processing related to the lighting of the light sources 22, such as determination of lighting patterns of the light sources 22.

The control circuit 30 is coupled to the detection circuit 15 via wiring 19 and obtains an output from the detection circuit 15. The control circuit 30 also controls the timing of obtaining the output from the detection circuit 15, that is, the timing of operating the scan circuit 14 so as to provide a gate signal to a scan line 6. Thus, the control circuit 30 controls operations of the light sources 22 and the planar optical sensor 10. The control circuit 30 further performs processing based on outputs of a plurality of optical sensors WA. Such processes include various types of processes, such as an outline extraction process and the Hough transform, which are to be described later. Such processes also include a determination process to determine whether a colony has been formed. Such a process will be described later.

Although not illustrated in the drawings, the detection device 1 includes an analog-to-digital conversion circuit, a digital-to-analog conversion circuit, and other components. The analog-to-digital conversion circuit is a circuit for allowing the outputs from the optical sensors WA (refer to FIG. 2) transmitted via the detection circuit 15 to be handled in arithmetic processing by the control circuit 30. The digital-to-analog conversion circuit is a circuit for allowing digital signals generated by the arithmetic processing of the control circuit 30 to be used for controlling the operations of the planar optical sensor 10 and the light source panel 20. These circuits may be included, for example, in part or in whole in the detection circuit 15. These circuits may alternatively be functions performed by circuits mounted on flexible printed circuits (FPCs) provided as the wiring 19 and the wiring 29. These circuits may alternatively be mounted in other ways on the detection device 1.

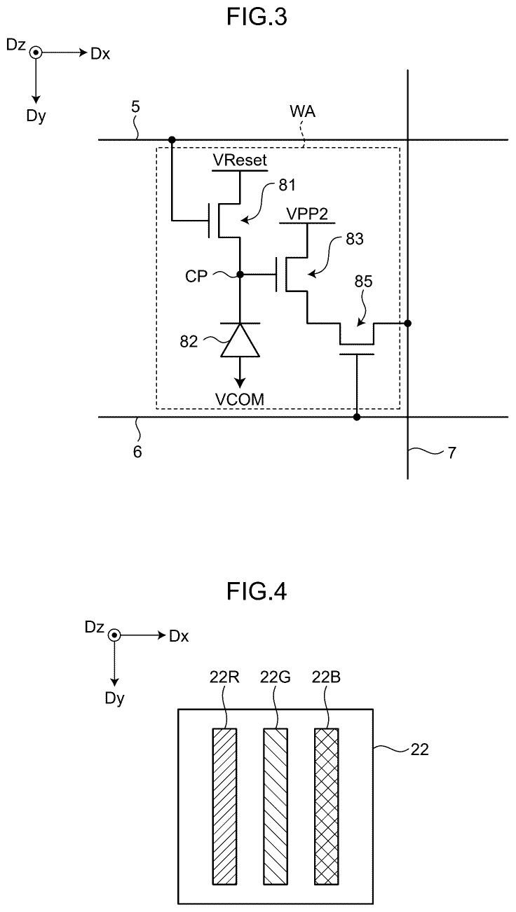

FIG. 2 is a diagram illustrating a configuration example of the detection area SA and the wiring area VA. A plurality of the optical sensors WA (FIG. 3) are two-dimensionally arranged in the detection area SA of the planar optical sensor 10. In the embodiment, as illustrated in FIG. 2, the optical sensors WA are arranged in a matrix having a row-column configuration along a first direction Dx and a second direction Dy. The first direction Dx is orthogonal to the second direction Dy. In the following description, the term “third direction Dz” refers to a direction orthogonal to the first direction Dx and the second direction Dy.

The reset circuit 13 is coupled to reset signal transmission lines 51, 52, . . . , 5n. Hereafter, the term “reset signal transmission line 5” refers to any one of the reset signal transmission lines 51, 52, . . . , 5n. The reset signal transmission line 5 is wiring along the first direction Dx. In the example illustrated in FIG. 2, n reset signal transmission lines 5 are arranged in the second direction Dy. n is a natural number equal to or larger than 2. The n reset signal transmission lines 5 are each coupled, at one end in the first direction Dx, to the reset circuit 13.

The scan circuit 14 is coupled to scan lines 61, 62, . . . , 6n. Hereafter, the term “scan line 6” refers to any one of the scan lines 61, 62, . . . , 6n. The scan line 6 is wiring along the first direction Dx. In the example illustrated in FIG. 2, n scan lines 6 are arranged in the second direction Dy. The n scan lines 6 are each coupled, at the other end in the first direction Dx, to the scan circuit 14.

As illustrated in FIG. 2, the reset signal transmission lines 5 and the scan lines 6 are alternately arranged in the second direction Dy in the detection area SA. The reset circuit 13 and the scan circuit 14 illustrated in FIGS. 1 and 2 are arranged at locations facing each other with the detection area SA interposed therebetween, but the layout of the reset circuit 13 and the scan circuit 14 is not limited to this layout and can be changed as appropriate.

Signal lines 71, 72, . . . , 7m are also provided in the detection area SA. Hereafter, the term “signal line 7” refers to any one of the signal lines 71, 72, . . . , 7m. The signal line 7 is wiring along the second direction Dy.

In the example illustrated in FIG. 2, m signal lines 7 are arranged in the first direction Dx. m is a natural number equal to or larger than 2. The m signal lines 7 are each coupled, at one end in the second direction Dy, to one of a plurality of switches (for example, a switch SW1, a switch SW2, a switch SW3, or a switch SW4) included in a multiplexer 40.

The multiplexer 40 is provided in the wiring area VA. The multiplexer 40 includes a plurality of switches. In the example illustrated in FIG. 2, the switches SW1, SW2, SW3, and SW4 are illustrated as the switches. The switches included in one multiplexer 40 are turned on (conducting state) at different times from one another. During a period when one of the switches included in one multiplexer 40 is on (conducting state), the other switches are off (non-conducting state). The number of the multiplexers 40 corresponds to the number (m) of the signal lines 7. When the number of the switches is p, m/p is sufficient as the number of the multiplexers 40. When more than one multiplexer 40 are provided, each of the multiplexers 40 is coupled to the detection circuit 15 via an individual one of wiring lines 401, 402, . . . , 40p.

The coupling between the signal lines 7 and the detection circuit 15 via the multiplexer 40 is merely exemplary and is not limited to this example. The signal lines 7 may be individually directly coupled to the detection circuit 15 in the wiring area VA. In the wiring area VA, the reset circuit 13 is coupled to the detection circuit 15 via wiring 131. In the wiring area VA, the scan circuit 14 is coupled to the detection circuit 15 via wiring 149.

In the detection of light by a photodiode (PD) 82 (refer to FIG. 3) provided in the optical sensor WA, the detection circuit 15 outputs signals to control operation timing of the reset circuit 13 and the scan circuit 14 under the control of the control circuit 30. The detection circuit 15 receives the outputs from the optical sensors WA. The detection circuit 15 converts signals received from the optical sensors WA into data that can be interpreted by the control circuit 30 and outputs the data to the control circuit 30. The detection circuit 15 of the embodiment is a microcontroller unit (MCU).

FIG. 3 is a circuit diagram illustrating a circuit configuration of the optical sensor WA. The first direction Dx and the second direction Dy in FIG. 3 merely correspond to the directions of the reset signal transmission lines 5, the scan lines 6, and the signal lines 7, and do not exactly indicate the relative positional relation of the circuit configuration in the optical sensor WA.

As illustrated in FIG. 3, a switching element 81, the PD 82, a transistor element 83, and a switching element 85 are provided in the optical sensor WA. The PD 82 is a photodiode (PD). The switching elements 81 and 85 and the transistor element are metal-oxide semiconductor field-effect transistors (MOSFETs).

The gate of the switching element 81 is coupled to the reset signal transmission line 5. One of the source and the drain of the switching element 81 is supplied with a reset potential VReset. The other of the source and the drain of the switching element 81 is coupled to the cathode of the PD 82 and the gate of transistor element 83. Hereafter, the term “coupling part CP” refers to a point where the other of the source and the drain of the switching element 81 is coupled to the cathode of the PD 82 and the gate of transistor element 83. A reference potential VCOM is supplied from the anode side of the PD 82. The potential difference between the reset potential VReset and the reference potential VCOM is set in advance, but the reset potential VReset and the reference potential VCOM may be variable. The reset potential VReset is higher than the reference potential VCOM.

The drain of the transistor element 83 serving as a source follower is supplied with an output source potential VPP2. The source of the transistor element 83 is coupled to one of the source and the drain of the switching element 85. The other of the source and the drain of the switching element 85 is coupled to the signal line 7. The gate of the switching element 85 is coupled to the scan line 6.

The reset potential VReset, the reference potential VCOM, and the output source potential VPP2 are supplied by the detection circuit 15 to the optical sensor WA based on, for example, electric power supplied via a power supply circuit (not illustrated) coupled to the detection circuit 15. The output form of these potentials is not limited to this form, and can be changed as appropriate.

The output source potential VPP2 is set in advance. The potential on the source side of the transistor element 83 is a potential lower than the output potential of the PD 82 by a voltage (Vth) between the gate and the source of the transistor element 83. In this case, the potential on the source side of the transistor element 83 corresponds to the reset potential VReset and the reference potential VCOM. The potential of the output of the PD 82 corresponds to photovoltaic power generated by the PD 82 in response to the light detected by the PD 82 during an exposure period.

When the gate of the switching element 85 is turned on by the gate signal supplied from the scan circuit 14 via the scan line 6, the source and the drain of the switching element 85 are brought into a conducting state therebetween. This operation transmits, to the signal line 7 via the switching element 85, a signal (potential) transmitted via the transistor element 83 to the switching element 85. Thus, the output from the optical sensor WA is generated. Hereinafter, the term “gate signal” refers to the signal (potential) supplied from the scan circuit 14 via the scan line 6. The scan circuit 14 is a circuit that outputs the gate signal. As described with reference to FIGS. 2 and 3, the optical sensors WA coupled to the scan lines 6 and the signal lines 7 are arranged in a matrix having a row-column configuration in the detection area SA of the planar optical sensor 10. The scan line 6 is provided along the first direction Dx and is configured to transmit the gate signal that causes the optical sensors WA to generate the outputs. The signal line 7 is configured to transmit the outputs of the optical sensors WA along the second direction Dy.

The output of one PD 82 provided in one optical sensor WA corresponds to the intensity of the light detected by the PD 82 during the exposure period set in advance. The output of the PD 82 is reset in response to a signal supplied by the reset circuit 13 via the reset signal transmission line 5. When the signal turns on the gate of the switching element 81, the source and the drain of the switching element 81 are brought into a conducting state therebetween. This operation resets the potential of the coupling part CP to the reset potential VReset.

FIG. 4 is a schematic diagram illustrating a configuration example of the light source 22. As illustrated in FIG. 4, the light source 22 includes a first light source 22R, a second light source 22G, and a third light source 22B. The first light source 22R, the second light source 22G, and the third light source 22B emit light in different colors from one another. In the embodiment, the first light source 22R emits red (R) light. The second light source 22G emits green (G) light. The third light source 22B emits blue (B) light.

In the light source 22 illustrated in FIG. 4, the longitudinal directions of the first light source 22R, the second light source 22G, and the third light source 22B are along the second direction Dy, and the first light source 22R, the second light source 22G, and the third light source 22B are arranged in this order from one side toward the other side in the first direction Dx. However, this arrangement is an exemplary form of the light source 22, and is not limited to this example. For example, the shapes of the first light source 22R, the second light source 22G, and the third light source 22B in the light source 22 as viewed from a planar viewpoint and the positional relation between the first light source 22R, the second light source 22G, and the third light source 22B can be changed as appropriate. The term “planar viewpoint” refers to a viewpoint from which a plane along the first direction Dx and the second direction Dy (Dx-Dy plane) is squarely viewed.

FIG. 5 is a schematic diagram schematically illustrating a configuration example of a detection system 100 including the detection device 1. As illustrated in FIG. 5, the detection system 100 includes a plurality of the detection devices 1, a host integrated circuit (IC) 70, and a coupling circuit 125. The detection devices 1 are electrically coupled to the common host IC 70 via the coupling circuit 125.

An incubator 120 illustrated in FIG. 5 is maintained such that an environment (temperature, humidity, and the like) therein is suitable for cultivation at an object to be detected 200 while a door is closed. The detection devices 1 are placed in the incubator 120.

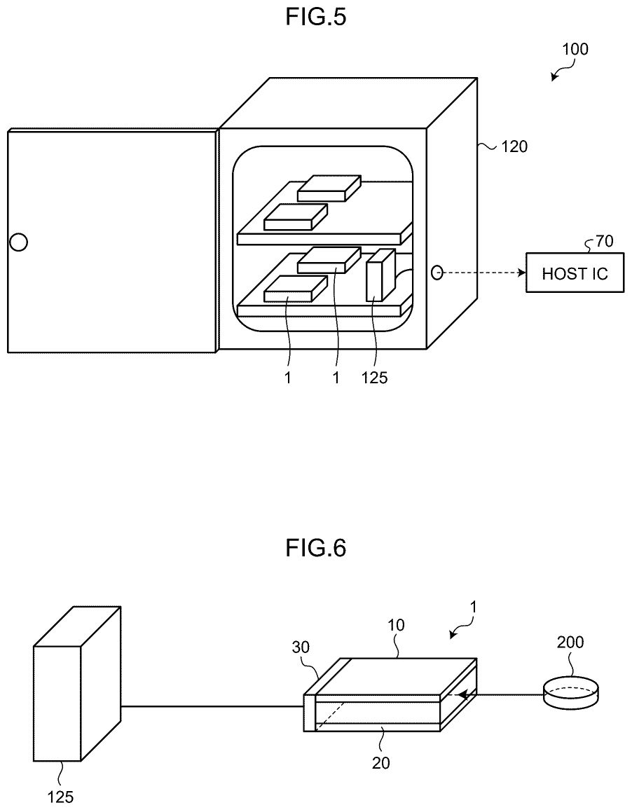

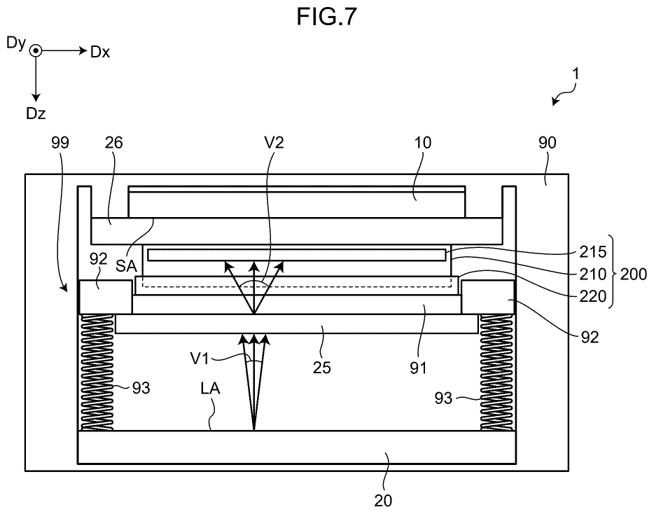

FIG. 6 is a schematic diagram illustrating a relation between one of the detection devices 1 and an external configuration. As illustrated in FIG. 6, the detection device 1 is coupled to the coupling circuit 125 by coupling the control circuit 30 to the coupling circuit 125. As illustrated in FIG. 6 and FIG. 7, which is to be described later, the planar optical sensor 10 faces the light source panel 20. The object to be detected 200 is placeable between the planar optical sensor 10 and the light source panel 20.

FIG. 6 only schematically illustrates a rough relation between the planar optical sensor 10, the light source panel 20, and the object to be detected 200. A specific structure for placing the object to be detected 200 between the planar optical sensor 10 and the light source panel 20 will be described with reference to FIG. 7.

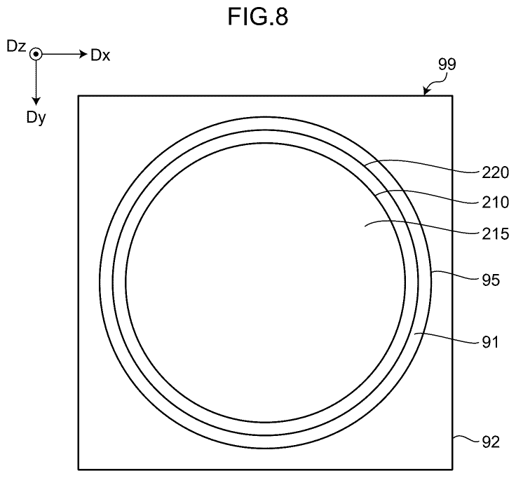

FIG. 7 is a schematic diagram illustrating main components of the detection device 1 and also illustrating structures of components including the object to be detected 200 placed on the detection device 1. The object to be detected 200 is a culture medium 215 (e.g., agar) accommodated in a dish 210 of a container. The container further includes a lid 220. The dish 210 is specifically a Petri dish. The lid 220 is a cover of the dish 210. As illustrated in FIG. 8 and other drawings to be explained later, the inner diameter of an annular sidewall of the lid 220 is equal to or greater than the outer diameter of the annular sidewall of the dish 210. That is, the lid 220 has a cylindrical outer circumferential wall that covers a cylindrical outer circumferential wall of the dish 210 from outside. The culture medium 215 is a culture medium on which colonies can be cultured. Hereinafter, the term simply called “colony” refers to a colony formed of cultivation targets that have been cultured on the culture medium 215 formed on the object to be detected 200. The cultivation targets are objects, such as biological tissues or microorganisms, that are assumed to be cultured on the culture medium 215. The culture medium 215 has a light-transmitting property, and the degree of light transmission thereof varies depending on the presence or absence of the colony and the thickness of the colony. The object to be detected 200 is placed on a light-transmitting member 91. The light-transmitting member 91 is a plate-like member made of colorless glass or a light-transmitting colorless synthetic resin.

FIG. 8 is a schematic plan view illustrating a case where the object to be detected 200 placed on the light-transmitting member 91 is viewed from the planar optical sensor 10 side. As illustrated in FIG. 8, the light-transmitting member 91 is a circular member having a diameter large enough to accommodate therein the object to be detected 200 as viewed from a planar viewpoint. The light-transmitting member 91 forms a light-transmitting area that can accommodate therein the object to be detected 200 between the planar optical sensor 10 and the light source panel 20. The light-transmitting member 91 is in contact with a light-blocking member 92 at the outer peripheral edge. The light-blocking member 92 is a plate-like member into which the light-transmitting member 91 is fitted. The light-blocking member 92 has a light-blocking property.

An edge 95 illustrated in FIG. 8 is the outer peripheral edge of the light-transmitting member 91 and is the inner peripheral edge of the light-blocking member 92 into which the light-transmitting member 91 is fitted. The edge 95 is circular as viewed from a planar viewpoint. A light-transmitting area may be formed inside the edge 95 by overlaying a light-transmitting member serving as the light-transmitting member 91 onto the light-blocking member 92 hollowed out in a circular shape so as to form an inner peripheral edge corresponding to the edge 95. In this case, the light-transmitting member 91 need not have a circular disc shape.

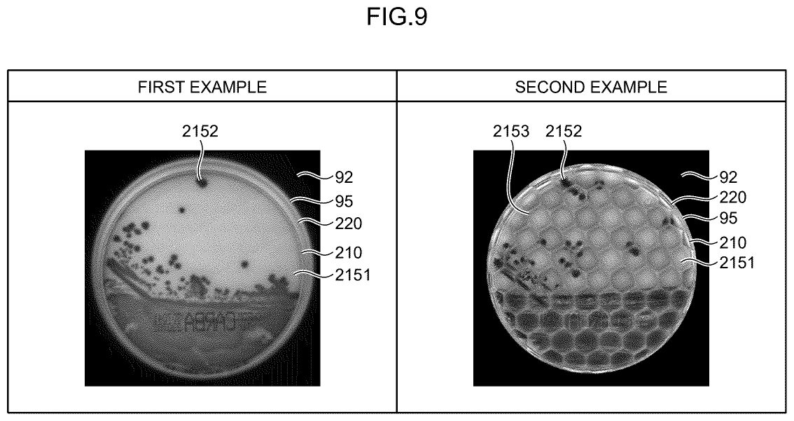

In the embodiment, a diffusion plate 25 is provided on the light source panel 20 side of the light-transmitting member 91. The diffusion plate 25 is an optical component that diffuses light. The diffusion plate 25 is located to be interposed between the light-transmitting member 91 and the light-emitting area LA of the light source panel 20. When the diffusion plate 25 receives the light emitted from the light-emitting area LA from the light source panel 20 side, the diffusion plate 25 further diffuses the traveling direction of the light as the light is transmitted toward the light-transmitting member 91. With this diffusion, the light from the light-emitting area LA formed by a set of the light sources 22 that are two-dimensionally arranged is made more uniform as viewed from a planar viewpoint, as illustrated in a first example in FIG. 9 to be explained later. The diffusion plate 25 of the embodiment serves as a dimmer (light controller) that is located between the light-transmitting member 91 of an object placement member 99 and the light sources 22 and diffuses the light from the light sources 22.

As illustrated in FIG. 7, in the embodiment, an elastic member 93 is provided between the light-blocking member 92 and the light source panel 20. The elastic member 93 has elasticity to urge the light-blocking member 92 toward the planar optical sensor 10. Specifically, the elastic member 93 is a cylindrical compression coil spring, as illustrated, for example, in FIG. 8. The object to be detected 200 placed on the light-transmitting member 91 is pressed against an optical member 26 provided between the planar optical sensor 10 and the light-transmitting member 91 by an urging force applied to the light-blocking member 92 by the elastic member 93. In the embodiment, an object placement member 99 is configured with the light-transmitting member 91, the light-blocking member 92, and the elastic member 93. In other words, the object placement member 99 includes the light-transmitting member 91 that is a light-transmitting member on which the object to be detected is placed, and the light-blocking member 92 that is a light-blocking member supporting the light-transmitting member from the outer periphery.

The optical member 26 serves as an optical member that limits the light that is emitted from the light-emitting area LA of the light source panel 20 and reaches the planar optical sensor 10. Specifically, the optical member 26 includes any one of a plate-shaped louver, a cylindrical opening, and a microlens. The plate-shaped louver has a plurality of plate-like structures arranged in parallel and having plate surfaces along the third direction Dz. The structures are preferably made of a material having a strong light-absorbing property. The optical member 26 is provided along a plane (Dx-Dy plane) orthogonal to the third direction Dz. The cylindrical opening penetrates the optical member 26 in the third direction Dz with respect to a base of the optical member 26. The base is preferably made of a material having a strong light-absorbing property. The microlens is a small lens having an optical axis along the third direction Dz. The base of the optical member 26 that supports the microlens is preferably made of a material having a strong light-absorbing property.

Regardless of what shape the optical member 26 has, the optical member 26 as the optical member is provided in order to limit the traveling direction of the light emitted from the light sources 22 and reaching the planar optical sensor 10 to the third direction Dz or a direction having a shallower inclination angle with respect to the third direction Dz. This configuration makes it easier to limit the area through which light to be detected by each of the optical sensors WA passes, to an area facing the optical sensor WA. The light before passing through the optical member 26 is affected by the scattering of light by the diffusion plate 25. Therefore, as will be described later with reference to FIG. 9, the light reaching the detection area SA is affected by the scattering of light by the diffusion plate 25, even if the optical member 26 is provided.

A housing 90 maintains a configuration in which the light-emitting area LA of the light source panel 20 and the detection area SA of the planar optical sensor 10 face in the third direction Dz. The housing 90 is a light-blocking housing provided so as to accommodate therein in advance the light source panel 20, the elastic member 93, the diffusion plate 25, the light-transmitting member 91, the light-blocking member 92, the optical member 26, and the planar optical sensor 10. Placing the object to be detected 200 between the optical member 26 and the light-transmitting member 91 establishes the positional relation among the components illustrated in FIG. 7. In the embodiment, the object to be detected 200 is placed on the light-transmitting member 91 so that the lid 220 side of the object to be detected 200 contacts the light-transmitting member 91. That is, the object to be detected 200 is placed between the planar optical sensor 10 and the light source panel 20 such that the lid 220 is located relatively below and the dish 210 is located relatively above.

As described above with reference to FIG. 7, the detection device 1 of the embodiment has a structure that allows the object to be detected 200 to be placed so as to be interposed between the planar optical sensor 10 and the light source panel 20. In the placed object to be detected 200, the bottom surface of the dish 210 with the culture medium 215 formed therein extends along the detection area SA of the planar optical sensor 10 and the light-emitting area LA of the light source panel 20.

The light emitted from the light-emitting area LA of the light source panel 20 is diffused by the diffusion plate 25, passes through the light-transmitting member 91, the object to be detected 200, and the optical member 26, and reaches the detection area SA of the planar optical sensor 10. Thus, the planar optical sensor 10 can be said to be configured to output data reflecting the intensity of light that has been emitted from the light source 22 and reached the optical sensors WA through the object to be detected 200. The data herein is data based on a set of the outputs from the optical sensors WA and can be regarded as data of an image. The image herein is obtained by regarding an output of one optical sensor WA as one pixel and arranging a plurality of the pixels so as to correspond to the arrangement of the optical sensors WA in the detection area SA. Hereinafter, the term simply called “image” refers to the set of the outputs of the optical sensors WA, unless otherwise noted. The term simply called “pixel” refers to the output of the optical sensor WA, unless otherwise noted. In practice, a process such as an analog-to-digital conversion is performed to regard the output of the optical sensor WA as the pixel. This process is performed by the detection circuit 15 in the embodiment as described above, but may be performed by the control circuit 30.

The intensity of the light reaching the detection area SA is affected by the degree of light transmission of the culture medium 215. The uniformity of the light reaching the detection area SA is affected by the degree of diffusion of the light between the planar optical sensor 10 and the light source panel 20.

FIG. 9 illustrates explanatory views regarding the effects of the light diffusion by the diffusion plate 25. The first example is an example of an image obtained in the embodiment. A second example is an example of an image obtained using a configuration that is obtained by removing the diffusion plate 25 from the embodiment. In other words, the first example is a diagram of a case where the light diffusion by the diffusion plate 25 occurs between the planar optical sensor 10 and the light source panel 20. The second example is a diagram of a case where the light diffusion by the diffusion plate 25 does not occur between the planar optical sensor 10 and the light source panel 20.

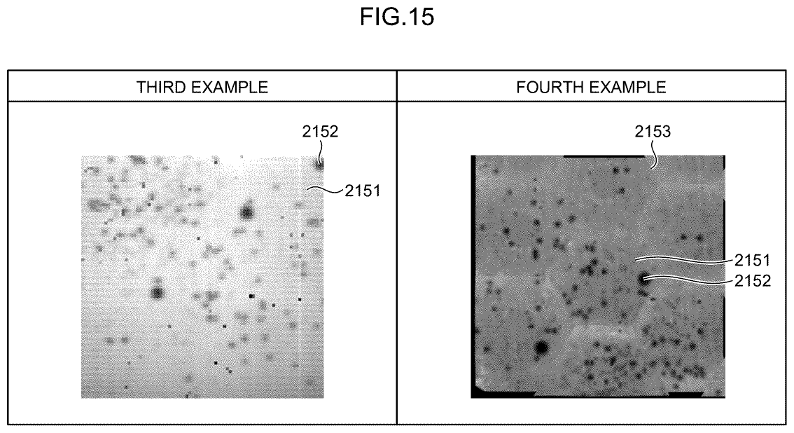

A bright area 2151 in FIG. 9 and in FIG. 15 to be explained later is formed by light that has passed through an area of the culture medium 215 where no colonies have formed. A dark area 2152 is formed by light that has passed through an area of the culture medium 215 where colonies have formed. A portion of the culture medium 215 in which colonies have formed is more difficult to transmit light than a portion of the culture medium 215 in which no colonies have formed. As a result, the dark area 2152 appears as relatively darker area than the bright area 2151. A pattern 2153 is a pattern generated by uneven light reaching the detection area SA. The pattern 2153 illustrated in FIGS. 9 and 15 appears as a honeycomb pattern, but patterns appearing as the pattern 2153 are not limited to this pattern. The pattern that appears as the pattern 2153 reflects a plurality of matters, such as the arrangement of the light sources 22 and the structure of the optical member 26, that affect the path of the light between the planar optical sensor 10 and the light source panel 20.

As described with reference to FIG. 7, in the embodiment, the diffusion plate 25 is provided between the planar optical sensor 10 and the light source panel 20. Therefore, the light from the light source 22 is diffused to a larger range of angle with respect to the third direction Dz and reaches the detection area SA. FIG. 7 illustrates the diffusion range of the light emitted from the light source 22 and before passing through the diffusion plate 25, roughly as a diffusion range V1. FIG. 7 also illustrates the diffusion range of the light emitted from the light source 22 and after passing through the diffusion plate 25, roughly as a diffusion range V2.

The diffusion of the light by the diffusion plate 25 produces an image with a clear difference in brightness between the bright area 2151 and the dark area 2152, as illustrated in the first example in FIG. 9 in the embodiment. Therefore, by comparing the image before the dark area 2152 occurs with the image after the dark area 2152 has occurred, the occurrence of the dark area 2152 and the degree of the occurrence of the dark area 2152 can be more easily determined. Thus, according to the embodiment, it is possible to obtain the image with which the degree of the formation of the colonies in the culture medium 215 can be excellently determined. That is, the diffusion of the light by the diffusion plate 25 creates the state that enables better detection of the colonies.

In contrast, without the diffusion of the light by the diffusion plate 25, an image in which the pattern 2153 appears is obtained as illustrated in the second example in FIG. 9. In the second example, a boundary between the bright area 2151 and the dark area 2152 is blurred in a portion of the image where the patterns 2153 appear. Therefore, in the second example, the detection of the degree of the formation of the colonies in the culture medium 215 is more difficult than in the first example.

In other words, the diffusion of the light by the diffusion plate 25 can reduce occurrence of the patterns 2153 in the image. That is, by providing the diffusion plate 25 between the planar optical sensor 10 and the light source panel 20, the image that allows better determination of the degree of formation of colonies in the culture medium 215 can be obtained.

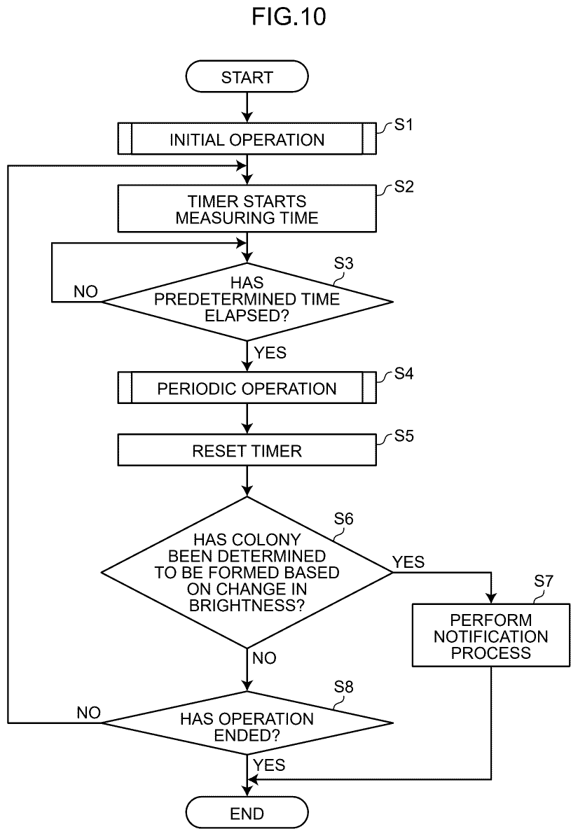

The following describes processing related to the operations of the detection device 1 with reference to flowcharts in FIGS. 10 to 12. Unless otherwise noted, in the embodiment, a process at each step illustrated in the flowcharts in FIGS. 10 to 12 is mainly performed by the control circuit 30.

FIG. 10 is a flowchart of processing related to the operations of the detection device 1. First, an initial operation is performed (Step S1). The time of the initial operation is immediately after the object to be detected 200 is placed on the detection device 1. That is, at the time of the initial operation, no colonies have been formed on the culture medium 215.

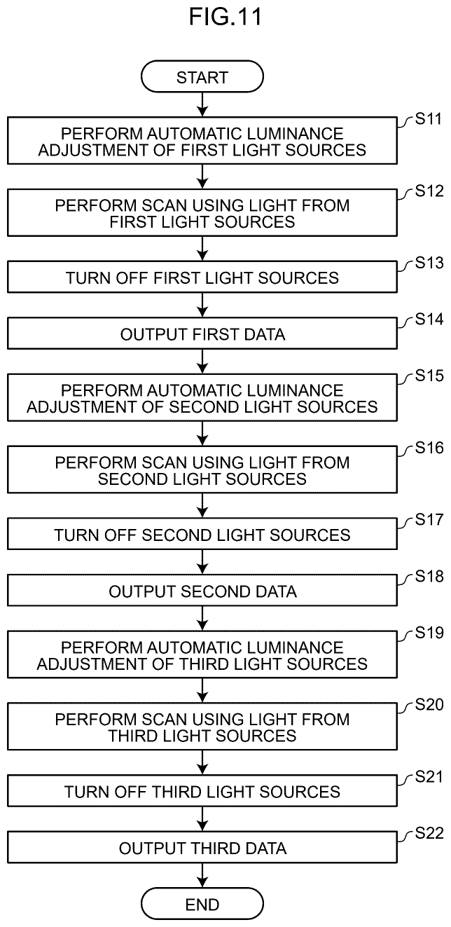

FIG. 11 is a flowchart of the initial operation. First, automatic luminance adjustment of the first light sources 22R is performed (Step S11). The automatic luminance adjustment in each of processes at Step S11 and at Steps S15 and S19 to be described later is a process to adjust the luminance levels of a plurality of light sources of the same color provided in the light-emitting area LA to pre-assumed luminance. The following describes an exemplary case in which the automatic luminance adjustment of a plurality of the first light sources 22R is performed in the process at Step S11. In this example, the operation is controlled such that the first light sources 22R start operating at either the lowest luminance or the highest luminance and change in luminance toward the other of the lowest luminance and the highest luminance with the lapse of time. During the passage of the time, the detection of the light using the optical sensors WA provided in the detection area SA and the output from the optical sensors WA are periodically performed. The luminance of the first light sources 22R is regarded as the pre-assumed luminance when the outputs of the optical sensors WA reach outputs corresponding to the pre-assumed luminance. The lowest luminance is the lower limit of an adjustable range of luminance of the light sources (such as the first light sources 22R). The adjustment of the luminance of the light sources within the adjustment range depends on the adjustment of the power supplied to the light sources. The adjustment range of the luminance and the adjustment range of the power corresponding thereto are preset. The highest luminance is the upper limit of the adjustable range of the luminance of the light sources (such as the first light sources 22R).

In the process at Step S11 in the embodiment, the luminance levels of the first light sources 22R are adjusted individually. Specifically, the first light source 22R is associated in advance with the optical sensor WA, the output of which reflects the luminance of the first light source 22R. That is, when the output of the optical sensor WA becomes an output corresponding to preset “target luminance to be achieved by the automatic luminance adjustment”, the automatic luminance adjustment of the first light source 22R associated with the optical sensor WA is completed. More specifically, each of the optical sensors WA detects light from the first light source 22R associated with the optical sensor WA more strongly than light from the other first light sources 22R. Therefore, the first light source 22R and the optical sensor WA associated with each other are arranged so as to overlap or substantially overlap each other as viewed from a planar viewpoint. The luminance of the first light source 22R is determined in this way, thus completing the automatic luminance adjustment.

In the process at each of Step S11 and Steps S15 and S19 to be described later, the control circuit 30 operates the planar optical sensor 10 and the light source panel 20 to perform the automatic luminance adjustment.

The description of a process at Step S15 to be described later is obtained by replacing the first light sources 22R in the description of the process at Step S11 with the second light sources 22G. The description of a process at Step S19 to be described later is obtained by replacing the first light sources 22R in the description of the process at Step S11 with the third light sources 22B. The specific process of the automatic luminance adjustment illustrated herein is only an example and is not limited to this example. The details may be changed as appropriate as long as the luminance of the multiple light sources of the same color can be set to the pre-assumed luminance as a result.

After the process at Step S11, a scan process using the light from the first light sources 22R is performed (Step S12). Specifically, the scan process is performed by the control circuit 30 operating the planar optical sensor 10 and the light source panel 20. In the process at Step S12, the light sources turned on by the operation of the light source panel 20 are the first light sources 22R. The second light sources 22G and the third light sources 22B are not turned on in the process at Step S12. As a result, the control circuit 30 obtains an image corresponding to the outputs of the optical sensors WA that have detected the light from the first light sources 22R transmitted through the object to be detected 200. At the completion of the process at Step S12, the first light sources 22R are turned off (Step S13).

In a process at Step S16 to be described later, the light sources to be turned on are not the first light sources 22R, but the second light sources 22G. In a process at Step S20 to be described later, the light sources to be turned on are not the first light sources 22R, but the third light sources 22B. After the processes at Steps S12 and S13, first data is output (Step S14). The first data is the image data obtained using the light from the first light sources 22R. Specifically, the control circuit 30 regards, as the first data, the image data reflecting the outputs of the optical sensors WA obtained in the process at Step S12.

After the process at Step S14, the automatic luminance adjustment of the second light sources 22G is performed (Step S15). After the process at Step S15, the scan process using the light from the second light sources 22G is performed (Step S16). Specifically, the scan process is performed by the control circuit 30 operating the planar optical sensor 10 and the light source panel 20. In the process at Step S16, the light sources turned on by the operation of the light source panel 20 are the second light sources 22G. The first light sources 22R and the third light sources 22B are not turned on in the process at Step S16. As a result, the control circuit 30 obtains an image corresponding to the outputs of the optical sensors WA that have detected the light from the second light sources 22G transmitted through the object to be detected 200. At the completion of the process at Step S16, the second light sources 22G are turned off (Step S17).

After the processes at Steps S16 and S17, second data is output (Step S18). The second data is the image data obtained using the light from the second light sources 22G. Specifically, the control circuit 30 regards, as the second data, the image data reflecting the outputs of the optical sensors WA obtained in the process at Step S16.

After the process at Step S18, the automatic luminance adjustment of the third light sources 22B is performed (Step S19). After the process at Step S19, the scan process using the light from the third light sources 22B is performed (Step S20). Specifically, the scan process is performed by the control circuit 30 operating the planar optical sensor 10 and the light source panel 20. In the process at Step S20, the light sources turned on by the operation of the light source panel 20 are the third light sources 22B. The first light sources 22R and the second light sources 22G are not turned on in the process at Step S20. As a result, the control circuit 30 obtains an image corresponding to the outputs of the optical sensors WA that have detected the light from the third light sources 22B transmitted through the object to be detected 200. At the completion of the process at Step S20, the third light sources 22B are turned off (Step S21).

After the processes at Steps S20 and S21, the third data is output (Step S22). The third data is the image data obtained using the light from the third light sources 22B. The control circuit 30 regards, as third data, the image data reflecting the outputs of the optical sensors WA obtained in the process of Step S20.

The initial operation ends with the completion of the process at the first Step S22. As illustrated in FIG. 10, after the initial operation that is the process at Step S1, the timer starts measuring time (Step S2). The process at Step S2 may be performed, for example, by a timer circuit provided in the control circuit 30, by setting a variable that serves as a counter and updating the counter based on an operating clock of the control circuit 30, or by other methods.

After the start of measuring time by the process at Step S2, a check is made to determine whether a predetermined time has elapsed (Step S3). Until the predetermined time elapses, the control circuit 30 waits (No at Step S3), without performing the next process. The predetermined time is five minutes, for example, but is not limited thereto. The predetermined time may be determined as appropriate according to a cycle (time interval) at which determination of the formation of colonies is to be made. When the predetermined time has elapsed after the process at Step S2 (Yes at Step S3), the periodic operation is performed (Step S4).

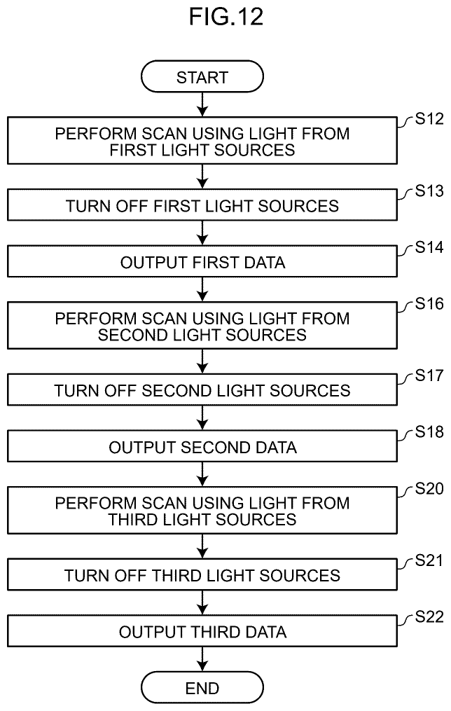

FIG. 12 is a flowchart of the periodic operation. The periodic operation is an operation in which the processes at Steps S11, S15, and S19 are omitted from the processes included in the initial operation described with reference to FIG. 11. In the periodic operation, the processes are performed in the following order: Step S12, Step S13, Step S14, Step S16, Step S17, Step S18, Step S20, Step S21, and Step S22.

The first light sources 22R, the second light sources 22G, and the third light sources 22B are turned on at different times. While one group of a group of the first light sources 22R, a group of the second light sources 22G, and a group of the third light sources 22B is on, the other two groups are not on. These light sources are periodically turned on in the order of the first light sources 22R, the second light sources 22G, and the third light sources 22B. These operations are indicated by the processes at Steps S12, S13, S16, S17, S20, and S21 in the initial operation and the periodic operation.

The luminance of the first light sources 22R that are turned on in the periodic operation is the luminance adjusted by the automatic luminance adjustment by the process at Step S11 in the initial operation. The luminance of the second light sources 22G that are turned on in the periodic operation is the luminance adjusted by the automatic luminance adjustment by the process at Step S15 in the initial operation. The luminance of the third light sources 22B that are turned on in the periodic operation is the luminance adjusted by the automatic luminance adjustment by the process at Step S19 in the initial operation.

The periodic operation ends with the completion of the process at Step S22 at the second and subsequent times. As illustrated in FIG. 10, after the periodic operation that is the process at Step S4, the timer is reset (Step S5). That is, the timer that started measuring time at Step S2 is reset in the process at Step S5.

The control circuit 30 determines whether colonies have been formed based on a change in brightness between the data obtained in the initial operation and the data obtained in the periodic operation (Step S6). Specifically, the control circuit 30 compares t-th data obtained in the initial operation with the t-th data obtained in the periodic operation. If a dark area not included in the t-th data obtained in the initial operation is included in the t-th data obtained in the periodic operation, the control circuit 30 determines that the dark area is due to colonies. The value of “t” in the t-th data is 1, 2, or 3. In a case where t is 1, the control circuit 30 compares the first data obtained in the initial operation with the first data obtained in the periodic operation. If a dark area not included in the first data obtained in the initial operation is included in the first data obtained in the periodic operation, the control circuit 30 determines that the dark area is due to colonies. The same interpretation can be made also for a case where t=2 or t=3. The control circuit 30 individually performs the determination for each of the case where t=1, the case where t=2, and the case where t=3. The time point at which the size of the dark area has become large enough to be regarded as the colonies is determined in advance and can be changed as appropriate depending on the size of the colonies at which a notification is to be made by a notification process to be described later.

In the embodiment, if a dark area considered to be a colony appears in one or more of a case where t=1, a case where t=2, and a case where t=3, it is regarded that a colony is determined to have been formed in the process at Step S6. However, specific conditions for such determination are not limited to this condition. If a dark area considered to be a colony appears in two or more or all three of the case where t=1, the case where t=2, and the case where t=3, a colony may be determined to have been formed in the process at Step S6. The process at Step S6 corresponds to the determination process to determine whether a colony is formed based on a comparison between a plurality of images obtained at different times.

If the process at Step S6 determines that a colony has been formed (Yes at Step S6), the notification process is performed (Step S7). In the notification process, a predetermined notification method is used to perform the notification. In the embodiment, the notification process is performed to send electronic mail indicating the formation of the colony to an electronic mail address of a manager of the object to be detected 200. The electronic mail and text to be sent via the electronic mail are set in advance. In the embodiment, for example, the control circuit 30 serves as a sender of the electronic mail, but is not limited to this method. As another example, the control circuit 30 may output, to an external information processing device, a signal that serves as an instruction for the external information processing device to send the electronic mail, or may use other methods. The form of the notification performed in the notification process is not limited to the sending of the electronic mail. For example, a voice output device such as a speaker may be operated to output predetermined “voice to notify that a colony has been formed” or other forms of notification may be used. The process at Step S7 corresponds to a process to make an output indicating that a colony has been formed if the colony is determined to have been formed.

If the process at Step S6 determines that no colonies have been formed (No at Step S6), the process at Step S2 is re-performed unless the detection device 1 has ended operating (No at Step S8). That is, the timer counts time again, and the periodic operation, the resetting of the timer, and determination of whether a colony has been formed are performed each time the predetermined time elapses. If the detection device 1 has ended operating in the process at Step S8 (Yes at Step S8) or after the process at Step S7 is performed, the processing related to the operations of the detection device 1 ends.

As described above, according to the embodiment, the detection device 1 includes the light sources (light sources 22) that emit light, the planar optical sensor (planar optical sensor 10) on which the optical sensors (optical sensors WA) that detect the light from the light sources are two-dimensionally arranged, the object placement member (object placement member 99) on which the object to be detected (object to be detected 200) is placed between the light sources and the planar optical sensor, the light controller (diffusion plate 25) that is provided between the object placement member and the light sources and diffuses the light from the light sources, and the optical member (optical member 26) that is provided between the object placement member and the planar optical sensor and limits the light reaching the optical sensor. With this configuration, the diffusion of the light by the diffusion plate 25 creates the state that enables better detection of the colonies. Thus, the embodiment allows better detection of the colonies.

Specifically, the optical member (optical member 26) includes any one of a plate-shaped louver, a cylindrical opening, and a microlens. This configuration makes it easier to limit the area through which light to be detected by each of the optical sensors (optical sensors WA) passes to an area facing the optical sensor.

### Modification

The following describes a modification of the embodiment that has a configuration partially different from that of the embodiment described above, with reference to FIGS. 13 to 17. In the description of the modification, the same components as those in the embodiment are denoted by the same reference numerals, and will not be described again.

FIG. 13 is a schematic diagram illustrating main components of a detection device 1A according to the modification and structures of components including the object to be detected 200 placed on the detection device 1A. As illustrated in FIG. 13, in the modification, the light-transmitting member 91 in the embodiment is replaced with a dimming panel (light control panel) 910. The dimming panel 910 is a component provided so as to be switchable between a state of serving in the same way as the diffusion plate 25 and a state of serving in the same way as the light-transmitting member 91 in the embodiment. In the modification illustrated in FIG. 13, the diffusion plate 25 is not provided because the dimming panel 910 can serve in the same way as the diffusion plate 25 in the embodiment.

FIG. 13 schematically illustrates the diffusion range of the light emitted from the light source 22 and before passing through the dimming panel 910, as a diffusion range V11. FIG. 13 also schematically illustrates the diffusion range of the light emitted from the light source 22 and after passing through the dimming panel 910 and diffused by the dimming panel 910, as a diffusion range V21. When the dimming panel 910 is in a diffusion mode, the diffusion range V21 occurs. The diffusion mode is a mode of diffusing light. FIG. 13 also schematically illustrates the diffusion range of the light emitted from the light source 22 and after passing through the dimming panel 910, as a diffusion range V31. When the dimming panel 910 is in a non-diffusion mode, the diffusion range V31 occurs. The non-diffusion mode is a mode of transmitting light while less diffusing the light than in the diffusion mode. The degree of light diffusion in the diffusion range V21 is larger than in the diffusion range V31.

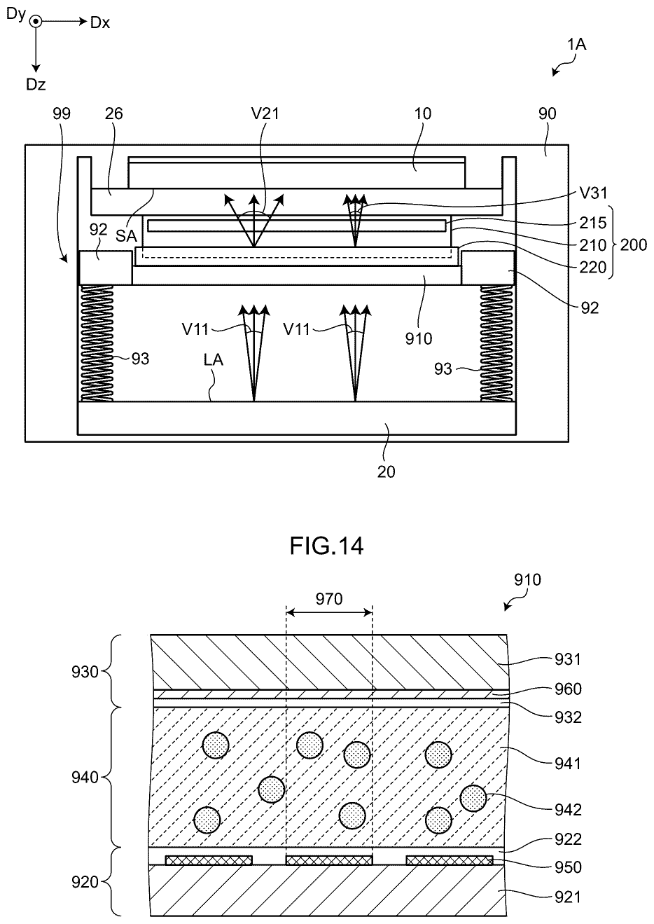

FIG. 14 is a schematic sectional view of the dimming panel 910. The dimming panel 910 includes a first substrate 920, a second substrate 930, and a liquid crystal 940.

The first substrate 920 includes a light-transmitting substrate 921, a pixel electrode 950, and an insulating layer 922. The pixel electrode 950 is individually provided in each pixel area 970, for example. The second substrate 930 includes a light-transmitting substrate 931, a common electrode 960, and an insulating layer 932.

In the present disclosure, the liquid crystal 940 is a polymer-dispersed liquid crystal (PDLC). That is, the dimming panel 910 is a liquid crystal panel enclosing the polymer-dispersed liquid crystal. Specifically, the liquid crystal 940 includes a bulk 941 and fine particles 942. The fine particles 942 change in orientation depending on a potential difference between the pixel electrode 950 and the common electrode 960 in the bulk 941. The light scattering state caused by the liquid crystal 940 is controlled to be switched by controlling the potential of the pixel electrode 950 for each pixel area 970.

FIG. 14 illustrates an example in which the pixel electrode 950 and the common electrode 960 are arranged so as to face each other with the liquid crystal 940 interposed therebetween. The dimming panel 910 may be configured such that the pixel electrode 950 and the common electrode 960 are provided on one substrate, and the orientation is changed by an electric field generated by the pixel electrode 950 and the common electrode 960, thus controlling the scattering state of the liquid crystal 940. In FIG. 14, the pixel electrode 950 is individually provided in each pixel area 970, but the specific form of the pixel electrode 950 is not limited to this configuration. The pixel electrode 950 may have a configuration continuous along the light-transmitting substrate 921 in the same way as the common electrode 960 that is continuous along the light-transmitting substrate 931. No matter what the specific configuration of the pixel electrode 950 is, the switching of the light scattering state caused by the liquid crystal 940 in the modification is evenly performed throughout the dimming panel 910. In other words, the switching of the light scattering state caused by the liquid crystal 940 in the modification is not performed on an individual pixel area 970 basis.

In the modification, when no potential difference is present between the pixel electrode 950 and the common electrode 960, the dimming panel 910 is placed in the non-diffusion mode (OFF) of substantially transmitting the light without diffusing it. In the modification, when a voltage is applied to the pixel electrode 950 so as to generate a potential difference between the pixel electrode 950 and the common electrode 960, the dimming panel 910 is placed in the diffusion mode (ON) of diffusing the light. Such control to switch the operating state of the dimming panel 910 is performed, for example, by the control circuit 30, but is not limited thereto, and a dedicated component for such control may be provided. The control circuit 30 of the modification controls the operations of the light source 22, the planar optical sensor 10, and the dimming panel 910, performs processing based on the outputs of the optical sensors WA.

FIG. 15 illustrates additional explanatory views regarding the effects of the light diffusion by the dimming panel 910. A third example illustrated in FIG. 15 is an enlarged view of a portion of the image of the dish 210 obtained when the dimming panel 910 is in the diffusion mode in which light is scattered. A fourth example is an enlarged view of a portion of the image of the dish 210 obtained when the dimming panel 910 is in the non-diffusion mode of transmitting the light.

In the third example, the patterns 2153 do not occur in the same way as in the first example, and the brightness of each of the bright areas 2151 and the dark areas 2152 is more pronounced than in the second and the fourth examples, thus making the determination regarding the degree of formation of colonies easier. In contrast, in the third example, the boundary between the bright area 2151 and the dark area 2152 is somewhat blurred.

In the fourth example, the pattern 2153 occurs in the same way as in the second example, but the boundary between the bright area 2151 and the dark area 2152 appears more clearly than in the third example. Therefore, when more accurate determination of the shape of the colony is required, it is advantageous to obtain an image similar to the fourth example.

Therefore, in the modification, until the colony is determined to have formed, the detection device 1A operates to obtain images while placing the dimming panel 910 in the diffusion mode. In the modification, after the colony is determined to have formed, the detection device 1A operates so as to obtain both an image while placing the dimming panel 910 in the diffusion mode and an image while placing the dimming panel 910 in the non-diffusion mode. This operation facilitates the operator to identify, from the image, the shape of the colony that have formed, after the colony is determined to have formed. Thus, the dimming panel 910 of the modification serves as the dimmer that is located between the light source 22 and the light-transmitting member 91 of the object placement member 99, and diffuses the light from the light source 22. The dimming panel 910 is provided so as to be switchable between the diffusion mode of diffusing the light and the non-diffusion mode of less diffusing the light than in the diffusion mode.

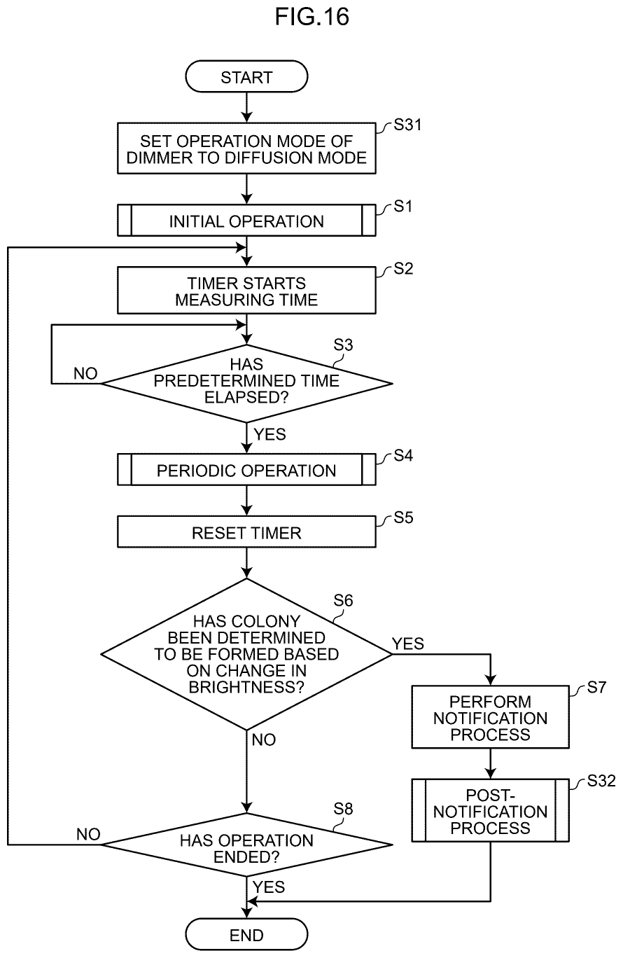

FIG. 16 is a flowchart of processing related to the operations of the detection device 1A according to the modification. In the description with reference to FIG. 16, matters different from the flow of the processing related to the operations of the detection device 1 described with reference to FIG. 10 will be specially described.

In the modification, as illustrated in FIG. 16, before a process at Step S1, a process is performed by the control circuit 30 to set the operation mode of the dimming panel 910 to the diffusion mode (Step S31). The process at Step S31 brings the dimming panel 910 into the state of diffusing the light. That is, before the initial operation by the process at Step S1, the dimming panel 910 is in a state of producing substantially the same optical effect as that of the diffusion plate 25 in the embodiment. Thus, the detection device 1A is operating in a state of being capable of obtaining the same image as the first example described with reference to FIG. 9. In this way, the control circuit 30 in the modification operates the dimming panel 910 in the diffusion mode until the colony is determined to have been formed.

In the modification, as illustrated in FIG. 16, after the process at Step S7, a post-notification operation is performed (Step S32).

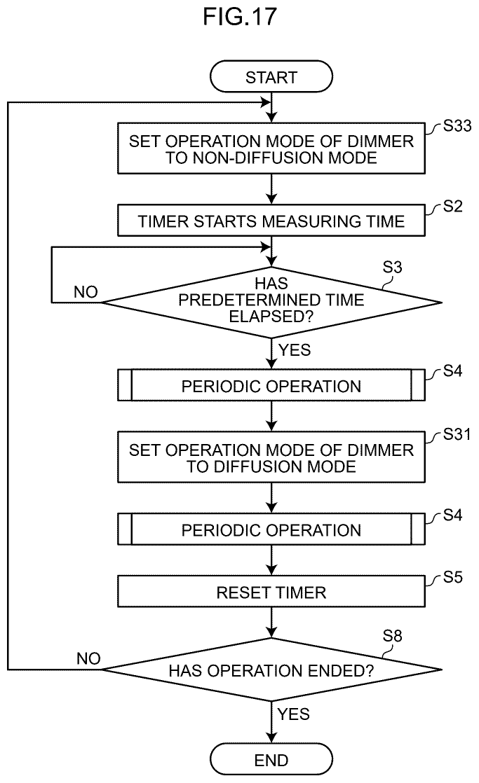

FIG. 17 is a flowchart of processing of the post-notification operation. In the description with reference to FIG. 17, the same process as that already described is assigned the same step number and will not be described in detail.

In the post-notification process, the control circuit 30 first sets the operation mode of the dimming panel 910 to the non-diffusion mode (Step S33). The process at Step S33 brings the dimming panel 910 into the state of substantially transmitting the light without diffusing it. After the process at Step S33, the process at Step S2, waiting for lapse of a predetermined time by the process at Step S3, and the process at Step S4 are sequentially performed. By performing the process at Step S33 before the periodic operation by the process at Step S4 at this point of time, the detection device 1A switches to a state of obtaining the same image as that of the fourth example described with reference to FIG. 15.

After the periodic operation by the process at Step S4 is completed in the state where an image similar to the fourth example described with reference to FIG. 15 is obtained, the process at Step S31 is performed. That is, the detection device 1A switches to a state of obtaining the same image as that of the first example described with reference to FIG. 9. After the process at Step S31, the periodic operation by the process at Step S4 is performed again. After the process at Step S4, the process at Step S5 is performed. After the process at Step S5, if the operation of the detection device 1A has not ended (No at Step S8), the process at Step S33 is performed. That is, the dimming panel 910 switches from the diffusion mode to the non-diffusion mode again, and the timer counts time, and each time the predetermined time elapses, the periodic operation, switching of the dimming panel 910 from the diffusion mode to the non-diffusion mode, the periodic operation, and resetting of the timer are performed. In the process at Step S8 after the process at Step S5, if the detection device 1A has ended operating (Yes at Step S8), the processing related to the operations of the detection device 1A ends, as illustrated in FIG. 16.

Except for the matters noted above, the detection device 1A is the same as the detection device 1 of the embodiment. After the colony is determined to have formed, the control circuit 30 of the modification alternately performs the processing to obtain the data while placing the dimming panel 910 in the diffusion mode and the processing to obtain the data while placing the dimming panel 910 in the non-diffusion mode. This operation is illustrated in the post-notification process.

As described above, according to the modification, the dimmer (light controller) is a light control panel (dimming panel 910) provided so as to be switchable between the diffusion mode of diffusing the light and the non-diffusion mode of less diffusing the light than in the diffusion mode. This configuration enables both better detection of the colonies by diffusing the light and more accurate confirmation of the shape of the colonies that have formed.

The modification further includes a processor (control circuit 30) that controls the operations of the light source (light source 22), the planar optical sensor (planar optical sensor 10), and the light control panel (dimming panel 910), and performs the processing based on the outputs of the optical sensors (optical sensors WA). The object to be detected (object to be detected 200) is the culture medium (culture medium 215) that is accommodated in the dish (dish 210) of the container. The planar optical sensor outputs the data reflecting the intensity of the light emitted from the light source and reaching the optical sensors through the object to be detected. The processor determines whether a colony has formed in the culture medium based on the comparison between a plurality of pieces of the data obtained at different times, and operates the light control panel (dimming panel 910) in the diffusion mode until a colony is determined to have formed. This operation enables the operation prioritizing better detection of a colony until a colony is determined to have formed.

In the modification, the processor (control circuit 30) makes an output indicating that a colony has formed if the colony is determined to have formed. After the colony is determined to have formed, the processor (control circuit 30) alternately performs the processing to obtain the data while placing the light control panel (dimming panel 910) in the diffusion mode and the processing to obtain the data while placing the light control panel in the non-diffusion mode. With this configuration, after the colony is determined to have formed, the processing can provide the data that allows more accurate identification of the shape of the colony that have formed.

In the modification, the light control panel (dimming panel 910) is the liquid crystal panel enclosing the polymer-dispersed liquid crystal. This configuration makes it easier to provide a configuration that enables both better detection of the colonies by diffusing the light and more accurate identification of the shape of the colonies that have formed.

Furthermore, as illustrated in FIG. 7, the object to be detected 200 is placed on the light-transmitting member 91 in the state where the dish 210 with the culture medium 215 formed therein is located relatively above and the lid 220 is located relatively below. With this configuration, even if condensation occurs in the dish 210, water droplets formed by the condensation can be more easily guided from an inner surface of the dish 210 to a gap between the dish 210 and the lid 220 and moved out of the dish 210.

In the embodiment, the light sources 22 including the first light sources 22R, the second light sources 22G, and the third light sources 22B are employed as light sources, but the light sources that can be employed in the embodiment according to the present disclosure are not limited to such light sources. For example, light sources corresponding to light in four or more colors of light may be employed, or light sources corresponding to one or two colors of light may be employed. Light in combined colors may also be used by simultaneously turning on some or all of a plurality of types of light sources that emit light in different colors. For example, when the first light sources 22R, the second light sources 22G, and the third light sources 22B are simultaneously turned on, white light is obtained. The light sources 22 may be simultaneously turned on, or the light sources 22 may be individually turned on at different times. The first example described with reference to FIG. 9 and the third example described with reference to FIG. 15 are images obtained in a planar light source mode in which the light sources 22 are turned on simultaneously on a color-by-color basis. The second example described with reference to FIG. 9 and the fourth example described with reference to FIG. 15 are images obtained in a point light source mode in which the light sources 22 are individually turned on at different times. The second and the fourth examples may both be obtained in the planar light source mode.

The vertical positional relation between the planar optical sensor 10 and the light source panel 20 is not limited to the example illustrated in FIG. 7, and may be reversed from the relation illustrated in FIG. 7. The elastic member 93 is not essential to the object placement member 99. For example, the light-blocking member 92 may be fixed to the housing 90 so that the light-transmitting member 91 is interposed between the planar optical sensor 10 and the light source panel 20. In this case, a gap allowing the object to be detected 200 to be inserted therein is provided above the light-transmitting member 91, between the light-transmitting member 91 and the planar optical sensor 10 or the light source panel 20.

Although the lid 220 is not essential in the object to be detected 200, the lid 220 is more preferably provided in order to reduce foreign matter entering the culture medium 215. The dish 210 of the embodiment is the Petri dish, but is not limited thereto, and may be other components that serve in the same way as the Petri dish.

Other operational advantages accruing from the aspects described in the present embodiment that are obvious from the description herein, or that are conceivable as appropriate by those skilled in the art will naturally be understood as accruing from the present disclosure.

What is claimed is:

1. A detection device comprising:
a light source configured to emit light;
a planar optical sensor on which a plurality of optical sensors configured to detect the light from the light source are two-dimensionally arranged;
an object placement member on which an object to be detected is placed between the light source and the planar optical sensor;
a light controller that is provided between the object placement member and the light source and is configured to diffuse the light from the light source; and
an optical member that is provided between the object placement member and the planar optical sensor and configured to limit light reaching the optical sensors.

2. The detection device according to claim 1, wherein the light controller is a light control panel provided so as to be switchable between a diffusion mode of diffusing the light and a non-diffusion mode of less diffusing the light than in the diffusion mode.

3. The detection device according to claim 2, further comprising a processor configured to control operations of the light source, the planar optical sensor, and the light control panel, and perform processing based on outputs of the optical sensors, wherein
the object to be detected is a culture medium accommodated in a dish of a container,
the planar optical sensor is configured to output data reflecting an intensity of the light that has been emitted from the light source and reached the optical sensors through the object to be detected, and
the processor is configured to:
determine whether a colony has formed in the culture medium based on comparison between a plurality of pieces of the data obtained at different times, and
operate the light control panel in the diffusion mode until a colony is determined to have formed.

4. The detection device according to claim 3, wherein
the processor is configured to:
make an output indicating that a colony has formed if the colony is determined to have formed, and
alternately perform, after the colony is determined to have formed, processing to obtain the data while placing the light control panel in the diffusion mode and processing to obtain the data while placing the light control panel in the non-diffusion mode.

5. The detection device according to claim 2, wherein the light control panel is a liquid crystal panel enclosing a polymer-dispersed liquid crystal.

6. The detection device according to claim 1, wherein the optical member comprises any one of a plate-shaped louver, a cylindrical opening, and a microlens.
